(19) United States
(12) Patent Application Publication
(10) Pub. No.: US 2006/0153777 A1
(43) Pub. Date: Jul. 13, 2006

(54) Aerosol formulation for inhalation containing an anticholinergic

(75) Inventor: Barbara Niklaus-Humke, Damscheid (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/326,226

(22) Filed: Jan. 4, 2006

(30) Foreign Application Priority Data
Jan. 12, 2005 (DE) 10 2005 001 297

(51) Int. Cl. A61K 31/4745; A61L 9/04

(52) U.S. Cl. 424/45; 514/291

(57) ABSTRACT

A pharmaceutical formulation comprising:
(a) an active substance consisting essentially of a compound of formula 1 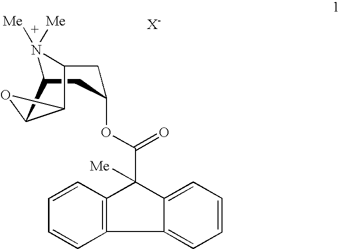
wherein X⁻ is an anion; (b) a solvent comprising ethanol or mixtures of ethanol and water; (c) at least one pharmacologically acceptable acid.

### RELATED APPLICATIONS

This application claims priority to German Application No. 10 2005 001 297.3, filed Jan. 12, 2005, which is hereby incorporated by reference in its entirety.

### FIELD OF THE INVENTION

The present invention relates to pharmaceutical preparations for inhalation containing as sole active substance a compound of formula 1

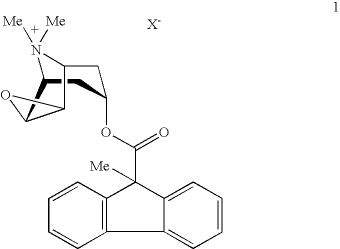

wherein X⁻ denotes an anion which is preferably selected from among chloride, bromide, iodide, sulfate, phosphate, methanesulfonate, nitrate, maleate, acetate, citrate, fumarate, tartrate, oxalate, succinate, benzoate, and p-toluenesulfonate; and

ethanol or mixtures of ethanol and water as the solvent, at least one pharmacologically acceptable acid and optionally other pharmacologically acceptable excipients and/or complexing agents.

The compounds of formula 1 are known from WO 03/064419, corresponding to U.S. Pat. Nos. 6,790,856 and 6,815,452, each of which is incorporated by reference herein. They have valuable pharmacological properties and may provide a therapeutic benefit as highly effective anticholinergics in the treatment of respiratory complaints, particularly in the treatment of inflammatory and/or obstructive respiratory complaints, particularly in the treatment of asthma or chronic obstructive pulmonary disease (COPD).

The present invention is concerned with liquid active substance formulations of these compounds for administration by inhalation, while the liquid formulations according to the invention must meet high quality standards. The formulations according to the invention may be inhaled through the mouth or nose. To achieve optimum distribution of the active substances in the lung, it makes sense to use a liquid formulation free from propellant gases which is administered using suitable inhalers. Such a formulation may be administered by inhalation by both oral and nasal route. Those inhalers which are capable of nebulizing a small amount of a liquid formulation in the dosage needed for therapeutic purposes within a few seconds into an aerosol suitable for therapeutic inhalation are particularly suitable. Within the scope of the invention, preferred nebulizers are those in which an amount of less than 100 microliters, preferably less than 50 microliters, most preferably less than 20 microliters of active substance solution can be nebulized preferably in one or two puffs to form an aerosol having an average particle size of less than 20 microns, preferably less than 10 microns, so that the inhalable part of the aerosol already corresponds to the therapeutically effective quantity.

An apparatus of this kind for the propellant-free administration of a metered amount of a liquid pharmaceutical composition for inhalation is described in detail, for example, in International Patent Application WO 91/14468 and also in WO 97/12687, cf FIGS. 6a and 6b and the accompanying description. In a nebulizer of this kind, a pharmaceutical solution is converted by means of a high pressure of up to 500 bar into an aerosol destined for the lungs, which is sprayed. WO 91/14468 is equivalent to U.S. Pat. Nos. 5,457,944 and 5,662,271; and WO 97/12687 is equivalent to U.S. Pat. Nos. 5,964,416, 6,402,055, and 6,497,373, all of which are hereby incorporated by reference.

In inhalers of this kind the formulations of solutions are stored in a reservoir. It is essential that the active substance formulations used are sufficiently stable when stored and at the same time are such that they can be administered directly, if possible without any further handling, in accordance with their medical purpose. Moreover, they must not contain any ingredients which might interact with the inhaler in such a way as to damage the inhaler or the pharmaceutical quality of the solution or of the aerosol produced.

To nebulize the solution, a special nozzle is used as described, for example, in WO 94/07607 or WO 99/16530. WO 94/07607 is equivalent to U.S. Pat. Nos. 5,911,851; 6,007,676; and 6,503,362, all of which are hereby incorporated by reference. WO 99/16530 is equivalent to U.S. Pat. No. 6,846,413, which is hereby incorporated by reference.

It is an aim of the present invention to provide a formulation of the compound of formula 1 which meets the high standards needed in order to be able to achieve optimum nebulization of a solution using the inhalers mentioned hereinbefore. The active substance formulations according to the invention must also be of sufficiently high pharmaceutical quality, i.e., they should be pharmaceutically stable over a storage time of some years, preferably at least one year, more preferably two years.

These propellant-free formulations of solutions must also be capable of being nebulized under pressure using an inhaler, the composition delivered by the aerosol formed falling reproducibly within a specified range.

Within the scope of the present invention it is preferable to use those compounds of formula 1 wherein the anion X⁻ is selected from among chloride, bromide, iodide, sulfate, phosphate, methanesulfonate, nitrate, maleate, acetate, citrate, fumarate, tartrate, oxalate, succinate, benzoate, and p-toluenesulfonate.

Preferably, salts of formula 1 are used wherein X⁻ denotes an anion selected from among the chloride, bromide, 4-toluenesulfonate, and methanesulfonate.

Particularly preferred within the scope of the present invention are those formulations which contain the compound of formula 1 wherein X⁻ denotes bromide.

References to the compound of formula 1 always include within the scope of the present invention all possible amorphous and crystalline modifications of this compound. References to the compound of formula 1 also include within the scope of the present invention all possible solvates and hydrates which may be formed from this compound.

Any reference to the compound 1′ which may be made within the scope of the present invention is to be regarded as a reference to the pharmacologically active cation of the following formula contained in the salts 1

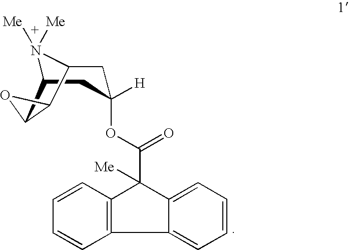

In the formulation according to the invention, the compound 1 is dissolved in ethanol or in mixtures of ethanol and water.

According to the invention, the formulation preferably contains only a single salt of formula 1. However, the formulation may also contain a mixture of different salts of formula 1. Formulations which contain active substances other than those of formula 1 are not covered by the invention.

The concentration of the compound of formula 1 based on the proportion of pharmacologically active cation 1′ in the pharmaceutical preparation according to the invention is about 0.1 to 1150 mg per 100 mL, according to the invention, preferably about 1 to 1000 mg per 100 mL. Particularly preferably, 100 mL of the formulations according to the invention contain about 5 to about 800 mg of 1′.

If the compound of formula 1 used is the particularly preferred compound wherein X⁻ denotes the bromide, the proportion of 1 according to the invention is about 0.1 to 1390 mg per 100 mL, preferably about 1.2 to 1210 mg per 100 mL of pharmaceutical preparation. Most preferably, 100 mL of the formulations according to the invention contain about 6 to 970 mg of 1.

Formulations according to the invention contain as solvent pure ethanol or mixtures of ethanol and water. If ethanol-water mixtures are used, the percentage of ethanol by mass in these mixtures is preferably in the range between 5% and 99% ethanol, particularly preferably in the range from 10% to 96% ethanol. Most particularly preferably within the scope of the invention, any ethanol-water mixtures used as solvent contain between 50% and 92%, particularly preferably between 69% and 91% ethanol.

Other cosolvents may optionally be used besides ethanol and water. However, it is preferable according to the invention not to use any additional solvent.

The formulations according to the invention contain pharmacologically acceptable inorganic or organic acids to adjust the pH. The pH of the formulations according to the invention is preferably kept within the range from 2.5 and 6.5, preferably between 3.0 and 5.0, particularly preferably between about 3.5 and 4.5.

Examples of preferred inorganic acids are selected from among hydrochloric acid, hydrobromic acid, nitric acid, sulfuric acid, and phosphoric acid. Examples of particularly suitable organic acids are selected from among ascorbic acid, citric acid, malic acid, tartaric acid, maleic acid, succinic acid, fumaric acid, acetic acid, formic acid, and propionic acid. Preferred inorganic acids are hydrochloric acid and sulfuric acid, while hydrochloric acid is of particular importance according to the invention. Of the organic acids, ascorbic acid, fumaric acid, and citric acid are preferred, while citric acid is particularly preferred according to the invention. If desired, mixtures of the abovementioned acids may also be used, particularly in the case of acids which have other properties in addition to their acidifying properties, e.g., those which act as flavorings or antioxidants, such as, for example, citric acid or ascorbic acid.

If desired, pharmacologically acceptable bases may be used to titrate the pH precisely. Suitable bases include for example alkali metal hydroxides and alkali metal carbonates. The preferred alkali metal ion is sodium. If bases of this kind are used, care must be taken to ensure that the resulting salts, which are then contained in the finished pharmaceutical formulation, are pharmacologically compatible with the abovementioned acid.

The formulations according to the invention may contain complexing agents as other ingredients. By complexing agents are meant within the scope of the present invention molecules which are capable of entering into complex bonds. Preferably, these compounds should have the effect of complexing cations, most preferably metal cations. The formulations according to the invention preferably contain edetic acid (EDTA) or one of the known salts thereof, e.g., sodium EDTA or disodium EDTA, as complexing agent. Preferably, sodium edetate is used, optionally in the form of its hydrates, more preferably in the form of its dihydrate.

If complexing agents are used within the formulations according to the invention, their content is preferably in the range from 0.1 to 3 mg per 100 mL, more preferably in the range from 0.2 to 2 mg per 100 mL of the formulation according to the invention. Particularly preferably, the formulations according to the invention contain a complexing agent in an amount of about 0.9 to 1.1 mg per 100 mL of the formulation according to the invention.

The remarks made concerning sodium edetate also apply analogously to other possible additives which are comparable to EDTA or the salts thereof, which have complexing properties and can be used instead of them, such as, for example, nitrilotriacetic acid and the salts thereof.

Other pharmacologically acceptable excipients may also be added to the formulation according to the invention. By adjuvants and additives are meant, in this context, any pharmacologically acceptable and therapeutically useful substance which is not an active substance, but can be formulated together with the active substance in the pharmacologically suitable solvent, in order to improve the qualities of the active substance formulation. Preferably, these substances have no pharmacological effects or no appreciable or at least no undesirable pharmacological effects in the context of the desired therapy. The adjuvants and additives include, for example, stabilizers, antioxidants, and/or preservatives which prolong the shelf life of the finished pharmaceutical formulation, as well as flavorings, vitamins and/or other additives known in the art. The additives also include pharmacologically acceptable salts such as sodium chloride, for example.

The preferred excipients include antioxidants such as ascorbic acid, for example, provided that it has not already been used to adjust the pH, vitamin A, vitamin E, tocopherols, and similar vitamins or provitamins occurring in the human body.

Preservatives can be added to protect the formulation from contamination with pathogenic bacteria. Suitable preservatives are those known from the prior art, particularly benzalkonium chloride or benzoic acid or benzoates such as sodium benzoate in the concentration known from the prior art. Preferably, benzalkonium chloride is added to the formulation according to the invention. The amount of benzalkonium chloride is between 1 mg and 50 mg per 100 mL of formulation, preferably about 7 to 15 mg per 100 mL, more preferably about 9 to 12 mg per 100 mL of the formulation according to the invention. However, formulations containing no preservatives are particularly preferred according to the invention.

Preferred formulations contain only benzalkonium chloride, disodium edetate and the acid needed to adjust the pH, in addition to ethanol or ethanol/water mixtures as solvent and the compounds of formula 1.

The pharmaceutical formulations according to the invention containing compounds of formula 1 are preferably used in an inhaler of the kind described hereinbefore in order to produce the propellant-free aerosols according to the invention. At this point we should once again expressly mention the patent documents described hereinbefore, to which reference is hereby made.

As described at the beginning, a further developed embodiment of the preferred inhaler is disclosed in WO 97/12687 (cf. in particular FIGS. 6a and 6b and the associated passages of description). This nebulizer (RESPIMAT®) can advantageously be used to produce the inhalable aerosols according to the invention. Because of its cylindrical shape and handy size of less than 9 to 15 cm long and 2 to 4 cm wide, the device can be carried by the patient at all times. The nebulizer sprays a defined volume of the pharmaceutical formulation out through small nozzles at high pressures, so as to produce inhalable aerosols.

The preferred atomizer essentially consists of an upper housing part, a pump housing, a nozzle, a locking clamp, a spring housing, a spring and a storage container, characterized by:
a pump housing fixed in the upper housing part and carrying at one end a nozzle body with the nozzle or nozzle arrangement,
a hollow piston with valve body,
a power take-off flange in which the hollow piston is fixed and which is located in the upper housing part,
a locking clamping mechanism located in the upper housing part,
a spring housing with the spring located therein, which is rotatably mounted on the upper housing part by means of a rotary bearing, and
a lower housing part which is fitted onto the spring housing in the axial direction.

The hollow piston with valve body corresponds to a device disclosed in WO 97/12687. It projects partially into the cylinder of the pump housing and is disposed to be axially movable in the cylinder. Reference is made particularly to FIGS. 1-4, especially FIG. 3, and the associated passages of description in the abovementioned International Patent Application. At the moment of release of the spring the hollow piston with valve body exerts, at its high pressure end, a pressure of 5 to 60 MPa (about 50 to 600 bar), preferably 10 to 60 MPa (about 100 to 600 bar) on the fluid, the measured amount of active substance solution. Volumes of 10 to 50 microliters are preferred, volumes of 10 to 20 microliters are more preferable, wherein a volume of 10 to 15 microliters per actuation is particularly preferred.

The valve body is preferably mounted at the end of the hollow piston which faces the nozzle body.

The nozzle in the nozzle body is preferably microstructured, i.e., produced by micro-engineering. Microstructured nozzle bodies are disclosed for example in WO 99/16530; reference is hereby made to the contents of this specification, especially FIG. 1 and the associated description. The nozzle body consists for example of two sheets of glass and/or silicon securely fixed together, at least one of which has one or more microstructured channels which connect the nozzle inlet end to the nozzle outlet end. At the nozzle outlet end there is at least one round or non-round opening 2 to 10 microns deep and 5 to 15 microns wide, the depth preferably being 4.5 to 6.5 microns and the length being 7 to 9 microns. If there is a plurality of nozzle openings, preferably two, the directions of spraying of the nozzles in the nozzle body may run parallel to each other or may be inclined relative to one another in the direction of the nozzle opening. In the case of a nozzle body having at least two nozzle openings at the outlet end, the directions of spraying may be inclined relative to one another at an angle of 20° to 160°, preferably at an angle of 60° to 150°, most preferably 80° to 100°. The nozzle openings are preferably arranged at a spacing of 10 to 200 microns, more preferably at a spacing of 10 to 100 microns, still more preferably 30 to 70 microns. A spacing of 50 microns is most preferred. The directions of spraying therefore meet in the region of the nozzle openings.

As already mentioned, the liquid pharmaceutical preparation hits the nozzle body at an entry pressure of up to 600 bar, preferably 200 to 300 bar and is atomized through the nozzle openings into an inhalable aerosol. The preferred particle sizes of the aerosol are up to 20 microns, preferably 3 to 10 microns.

The locking clamping mechanism contains a spring, preferably a cylindrical helical compression spring as a store for the mechanical energy. The spring acts on the power take-off flange as a spring member the movement of which is determined by the position of a locking member. The travel of the power take-off flange is precisely limited by an upper stop and a lower stop. The spring is preferably tensioned via a stepping-up gear, e.g., a helical sliding gear, by an external torque which is generated when the upper housing part is turned relative to the spring housing in the lower housing part. In this case, the upper housing part and the power take-off flange contain a single- or multi-speed spline gear.

The locking member with the engaging locking surfaces is arranged in an annular configuration around the power take-off flange. It consists, for example, of a ring of plastics or metal which is inherently radially elastically deformable. The ring is arranged in a plane perpendicular to the axis of the atomizer. After the locking of the spring, the locking surfaces of the locking member slide into the path of the power take-off flange and prevent the spring from being released. The locking member is actuated by means of a button. The actuating button is connected or coupled to the locking member. In order to actuate the locking clamping mechanism the actuating button is moved parallel to the annular plane, preferably into the atomizer, and the deformable ring is thereby deformed in the annular plane. Details of the construction of the locking clamping mechanism are described in WO 97/20590. WO 97/20590 is equivalent to U.S. Pat. No. 6,176,442, each of which is hereby incorporated by reference.

The lower housing part is pushed axially over the spring housing and covers the bearing, the drive for the spindle and the storage container for the fluid.

When the atomizer is operated, the upper part of the housing is rotated relative to the lower part, the lower part taking the spring housing with it. The spring meanwhile is compressed and biased by means of the helical sliding gear, and the clamping mechanism engages automatically. The angle of rotation is preferably a whole-number fraction of 360°, e.g., 180°. At the same time as the spring is tensioned, the power take-off component in the upper housing part is moved along by a given amount, the hollow piston is pulled back inside the cylinder in the pump housing, as a result of which some of the fluid from the storage container is sucked into the high pressure chamber in front of the nozzle.

If desired, a plurality of replaceable storage containers containing the fluid to be atomized can be inserted in the atomizer one after another and then used. The storage container contains the aqueous aerosol preparation according to the invention.

The atomizing process is initiated by gently pressing the actuating button. The clamping mechanism then opens the way for the power take-off component. The biased spring pushes the piston into the cylinder in the pump housing. The fluid emerges from the nozzle of the atomizer in the form of a spray.

Further details of the construction are disclosed in PCT applications WO 97/12683 (equivalent to U.S. Pat. No. 6,176,442) and WO 97/20590, which are each incorporated by reference. The components of the atomizer (nebulizer) are made of a material suitable for their function. The housing of the atomizer and, if the function allows, other parts as well are preferably made of plastics, e.g., by injection molding. For medical applications, physiologically acceptable materials are used.

FIGS. 6a/b of WO 97/12687 show the RESPIMAT(G) nebulizer with which the aqueous aerosol preparations according to the invention can advantageously be inhaled.

FIG. 6a shows a longitudinal section through the atomizer with the spring under tension, FIG. 6b shows a longitudinal section through the atomizer with the spring released.

The upper housing part (51) contains the pump housing (52), on the end of which is mounted the holder (53) for the atomizer nozzle. In the holder is the nozzle body (54) and a filter (55). The hollow piston (57) fixed in the power take-off flange (56) of the locking clamping mechanism projects partly into the cylinder of the pump housing. At its end the hollow piston carries the valve body (58). The hollow piston is sealed off by the gasket (59). Inside the upper housing part is the stop (60) on which the power take-off flange rests when the spring is relaxed. Located on the power take-off flange is the stop (61) on which the power take-off flange rests when the spring is under tension. After the tensioning of the spring, the locking member (62) slides between the stop (61) and a support (63) in the upper housing part. The actuating button (64) is connected to the locking member. The upper housing part ends in the mouthpiece (65) and is closed off by the removable protective cap (66).

The spring housing (67) with compression spring (68) is rotatably mounted on the upper housing part by means of the snap-fit lugs (69) and rotary bearings. The lower housing part (70) is pushed over the spring housing. Inside the spring housing is the replaceable storage container (71) for the fluid (72) which is to be atomized. The storage container is closed off by the stopper (73), through which the hollow piston projects into the storage container and dips its end into the fluid (supply of active substance solution).

The spindle (74) for the mechanical counter is mounted on the outside of the spring housing. The drive pinion (75) is located at the end of the spindle facing the upper housing part. On the spindle is the slider (76).

The nebulizer described above is suitable for nebulizing the aerosol preparations according to the invention to form an aerosol suitable for inhalation.

In another preferred embodiment the pharmaceutical formulation according to the invention is applied with the nebulizer described hereinbefore wherein a replaceable storage container is used which contains the pharmaceutical formulation according to the invention inside a gas- and fluid-tight container as described in WO 99/43571.

Some details of the construction of this container will now be given, the reference numerals quoted in the following description corresponding to those used in WO 99/43571. The description that follows thus makes reference to the entirety of the disclosure of WO 99/43571, corresponding to U.S. Pat. No. 6,685,691, each of which is incorporated by reference herein.

Accordingly, the formulations according to the invention may particularly preferably be administered using a gas- and fluid-tight container as a replaceable cartridge for a medicinal liquid in a propellant-free atomizer which comprises, as disclosed in WO 99/43571, a discharge outlet in the form of a hollow piston, the container comprising
a foil bag (11, 21, 31), which is closed off at both ends, at least one end being closed off by a weld seam (13, 23, 32) extending substantially at right angles to the axis of the bag and the foil bag being deformable by external pressure at a differential pressure between the interior of the container and its surroundings of below 300 hPa (300 mbar),
a dimensionally stable flange (15, 25, 34), tightly attached to the foil bag and designed as a releasable connecting member for fitting the container onto a discharge outlet (67),
a guide channel (42, 54) in the flange,
while in the guide channel is provided a sealing point (56, 64, 74) and/or a press fit (55, 66, 77), which surrounds the discharge outlet, and
a removal point for the liquid in the region of the guide channel into which the hollow piston penetrates during use so as to dip into the medicinal liquid.

If the formulation according to the invention is nebulized using the method described above (RESPIMAT®), the mass expelled, in at least 97%, preferably at least 98% of all the actuations of the inhaler (puff or puffs), should correspond to a defined quantity with a range of tolerance of not more than 25%, preferably 20% of this quantity. Preferably, between 5 and 30 mg, more preferably between 5 and 20 mg of formulation are delivered as a defined mass per puff.

However, the formulation according to the invention can also be nebulized using inhalers other than those described above, for example jet-stream inhalers.

The present invention also relates to an inhalation kit consisting of one of the pharmaceutical preparations according to the invention described above and an inhaler suitable for nebulizing this pharmaceutical preparation. The present invention preferably relates to an inhalation kit consisting of one of the pharmaceutical preparations according to the invention described above and the RESPIMAT® inhaler described above.

The examples of formulations given below serve as illustrations without restricting the subject matter of the present invention to the compositions shown by way of example.

### I. EXAMPLES OF FORMULATIONS

100 mL of Pharmaceutical Preparation Contains:

| | 1 | | disodium | | made up to 100 mL |
|---|---|---|---|---|---|
| | (1′-cation) | benzalkonium | edetate | citric acid | with ethanol/water |
| Example | (mg) | chloride (mg) | dihydrate (mg) | (mg) | mixture (% m/m) |
| 1 | 1100 | 10 | 3 | 40 | 50/50 |
| 2 | 500 | 10 | 3 | 10 | 50/50 |
| 3 | 9 | — | 3 | 3 | 70/30 |
| 4 | 90 | — | 2 | 3 | 70/30 |
| 5 | 725 | — | 1 | 3 | 70/30 |
| 6 | 1100 | — | 1 | 2 | 90/10 |
| 7 | 800 | — | 1 | 4 | 90/10 |
| 8 | 500 | — | 1 | 5 | 90/10 |
| 9 | 200 | 5 | — | 3 | 95/5 |
| 10 | 50 | 5 | — | 3 | 95/5 |
| 11 | 0.1 | 5 | — | 3 | 95/5 |

The formulations according to the invention are prepared analogously to methods known in the art, for example, by dissolving the formulation ingredients in the solvent ethanol or ethanol/water.

We claim:

1. A pharmaceutical formulation comprising:
(a) an active substance consisting essentially of a compound of formula 1 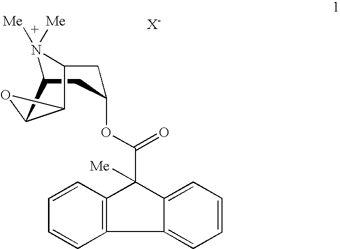
wherein X⁻ is an anion;
(b) a solvent comprising ethanol or mixtures of ethanol and water; and
(c) at least one pharmacologically acceptable acid.

2. The pharmaceutical formulation according to claim 1, wherein X⁻ is chloride, bromide, iodide, sulfate, phosphate, methanesulfonate, nitrate, maleate, acetate, citrate, fumarate, tartrate, oxalate, succinate, benzoate, orp-toluenesulfonate.

3. The pharmaceutical formulation according to claim 2, wherein X⁻ is chloride, bromide, methanesulfonate, or p-toluenesulfonate.

4. The pharmaceutical formulation according to claim 1, further comprising an additional pharmacologically acceptable excipient or complexing agent.

5. The pharmaceutical formulation according to claim 1, wherein the solvent is a mixture of ethanol and water where the proportion of ethanol by mass is in the range from 5% to 99%.

6. The pharmaceutical formulation according to claim 5, wherein the proportion of ethanol by mass in the solvent is in the range from 10% to 96%.

7. The pharmaceutical formulation according to claim 1, wherein the pharmacologically acceptable acid is hydrochloric acid, hydrobromic acid, nitric acid, sulfuric acid and phosphoric acid or from the organic acids ascorbic acid, citric acid, malic acid, tartaric acid, maleic acid, succinic acid, fumaric acid, acetic acid, formic acid, or propionic acid.

8. The pharmaceutical formulation according to claim 1, wherein the pH is 2.5 to 6.5.

9. The pharmaceutical formulation according to claim 1, wherein the amount of the cation of formula 1 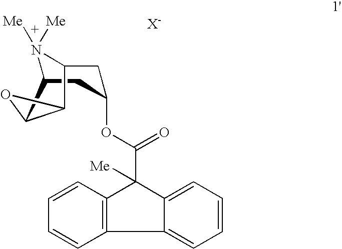
is about 0.1 to 1150 mg per 100 mL of solution.

10. The pharmaceutical formulation according to claim 1, further comprising a complexing agent.

11. The pharmaceutical formulation according to claim 10, wherein the amount of complexing agent is 0.1 to 3 mg per 100 mL of the pharmaceutical formulation.

12. The pharmaceutical formulation according to claim 1, further comprising benzalkonium chloride.

13. The pharmaceutical formulation according to claim 12, wherein the amount of benzalkonium chloride is 1 to 50 mg per 100 mL of the pharmaceutical formulation.

14. An inhalation kit comprising a nebulizer containing a pharmaceutical formulation according to claim 1.

15. The inhalation kit according to claim 14, wherein the nebulizer is a nebulizer according to WO 91/14468 or FIGS. 6a and 6b of WO 97/12687.
